SEQUENCE LISTING (1 sequence)

SEQ ID NO: 1 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

(19) United States
(12) Patent Application Publication
(10) Pub. No.: US 2026/0240815 A1
(43) Pub. Date: Aug. 20, 2026

(54) POTENT TRANSTHYRETIN (TTR) STABILIZATION IN TTR AMYLOIDOSIS PATIENTS RECEIVING ACORAMIDIS

(71) Applicant: EIDOS THERAPEUTICS, INC., Palo Alto, CA (US)

(72) Inventors: Uma SINHA, Palo Alto, CA (US); Alan X. JI, Palo Alto, CA (US); Paul WONG, Palo Alto, CA (US)

(21) Appl. No.: 19/159,890

(22) PCT Filed: Feb. 29, 2024
(86) PCT No.: PCT/US2024/017918

§ 371 Date: Aug. 26, 2025

Related U.S. Application Data
(60) Provisional application No. 63/487,801, filed on Mar. 1, 2023.
(60) Provisional application No. 63/516,757, filed on Jul. 31, 2023.

(51) Int. Cl. A61K 31/415; A61K 31/423; A61P 25/02

(52) U.S. Cl. CPC A61K 31/415; A61K 31/423; A61P 25/02

(57) ABSTRACT

Described herein are methods for treating transthyretin (TTR) amyloidosis with Compound 1 in a subject comprising certain mutations in the TTR protein (Compound 1). The methods include specific dosing regimens that have great efficacy in treating the subjects and that are well tolerated in subjects.

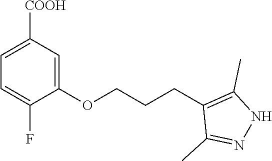 [SMILES: Cc1n[nH]c(C)c1CCCOc1cc(C(=O)O)ccc1F]

### CROSS-REFERENCES TO RELATED APPLICATIONS

This application claims the benefit of priority under 35 U.S.C § 119 (e) to U.S. Provisional Application Ser. No. 63/487,801 filed Mar. 1, 2023, and 63/516,757 filed Jul. 31, 2023, the disclosure of each are incorporated herein by reference in their entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

This application hereby incorporates by reference, in its entirety, the sequence listing created Feb. 27, 2024, 4,096 bytes, submitted in computer readable form with this application, entitled: 051418-510001WO.xml.

### BACKGROUND OF THE INVENTION

Aberrant protein interaction and aggregation, either through protein misfolding or over activation of a signaling pathway is the underlying cause of a large number of human degenerative diseases. As such, targeting protein protein interactions (PPIs) is of therapeutic interest.

One such example of aberrant protein aggregation is the soluble protein transthyretin (TTR or prealbumin). TTR is a 55 kDa homotetrameric protein present in blood and cerebrospinal fluid. When dissociated from its homotetrameric form, TTR dimers can misfold into amyloidogenic monomers. This has been observed with the wild type TTR as well as more than 100 different mutated variants. Research has shown that stabilizing the tetrameric form of TTR inhibits the misfolding of amyloidogenic monomers and subsequent TTR amyloid formation and deposition.

A benzoxazole derivative called Tafamidis (2-(3,5-dichlorophenyl)-1,3-benzoxazole-6-carboxylic acid) has been described to inhibit abnormal TTR aggregation and fibril formation. Tafamidis was approved by the U.S. Food and Drug Administration (FDA) in May of 2019 as the first approved pharmacotherapy for the treatment of the cardiomyopathy of wild type or hereditary TTR-mediated amyloidosis in adults to reduce cardiovascular mortality and cardiovascular-related hospitalization.

Acoramidis (3-(3-(3,5-dimethyl-1H-pyrazol-4-yl) propoxy)-4-fluorobenzoic acid, also known as AG10) is another compound under development for the treatment of TTR amyloid related diseases. This compound is a highly potent stabilizer of the TTR tetramer, but it is not yet approved by the FDA.

As such, there exists a need in the art to provide methods for treating abnormal TTR aggregation and fibril formation using acoramidis. The present disclosure addresses these needs and provides related advantages as well.

### SUMMARY

In some aspects, provided herein are methods of treating transthyretin (TTR) amyloidosis in a subject in need thereof, the method comprising administering to a subject in need thereof a therapeutically effective amount of Compound 1, having the formula:

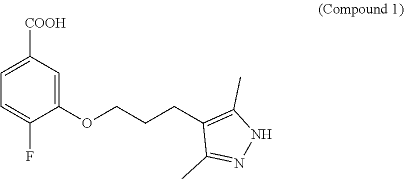 [SMILES: Cc1n[nH]c(C)c1CCCOc1cc(C(=O)O)ccc1F]

or a pharmaceutically acceptable salt thereof, wherein said therapeutically effective amount is a total daily dosage of about 10 milligrams (mg) to about 2,000 mg of Compound 1 in HCl salt form or an equivalent amount of Compound 1 in free base form or in a different salt form.

In some embodiments, the TTR amyloidosis in the subject is characterized by a TTR protein comprising a mutation selected from the group consisting of
a glycine to serine mutation at position 6 (G6S),
an alanine to serine mutation at position 25 (A25S),
a valine to methionine mutation at position 30 (V30M)
an alanine to aspartic acid mutation at position 36 (A36D),
a glutamic acid to aspartic acid mutation at position 42 (E42D),
a serine to arginine mutation at position 50 (S50R),
a threonine to alanine mutation at position 60 (T60A)
an isoleucine to leucine mutation at position 68 (168L),
a glutamic acid to glutamine mutation at position 89 (E89Q),
a glutamic acid to glutamine mutation at position 92 (E92Q),
a valine to leucine mutation at position 94 (V94L),
a valine to isoleucine mutation at position 122 (V122I),
an alanine to serine mutation at position 97 (A97S),
an aspartic acid to alanine mutation at position 38 (D38A),
a phenylalanine to leucine mutation at position 64 (F64L),
a leucine to histidine mutation at position 58 (L58H),
a proline to serine mutation at position 24 (P24S), and
a tyrosine to cysteine mutation at position 114 (Y114C).

In some embodiments, the TTR amyloidosis in the subject is characterized by a TTR protein comprising a mutation selected from the group consisting of
a glycine to serine mutation at position 6 (G6S),
an alanine to serine mutation at position 25 (A25S),
an alanine to aspartic acid mutation at position 36 (A36D),
a glutamic acid to aspartic acid mutation at position 42 (E42D),
a serine to arginine mutation at position 50 (S50R),
an isoleucine to leucine mutation at position 68 (168L),
a glutamic acid to glutamine mutation at position 89 (E89Q),
a glutamic acid to glutamine mutation at position 92 (E92Q),
a valine to leucine mutation at position 94 (V94L), and
an alanine to serine mutation at position 97 (A97S).

In some embodiments, the total daily dosage of Compound 1, or a pharmaceutically acceptable salt thereof is about 800 mg. In some embodiments, the total daily dosage of Compound 1, or a pharmaceutically acceptable salt thereof is about 1,500 to about 1,700 mg. In some embodiments, the total daily dosage of Compound 1, or a pharmaceutically acceptable salt thereof is about 1,600 mg. In some embodiments, the total daily dosage of Compound 1, or a pharmaceutically acceptable salt thereof is about 1,100 mg to about 1,300 mg. In some embodiments, the total daily dosage of Compound 1, or a pharmaceutically acceptable salt thereof is about 1,236 mg. In some embodiments, the HCl salt form of Compound 1 is administered.

In some embodiments, the total daily dosage is about 800 mg of Compound 1 in HCl salt form or an equivalent amount of Compound 1 in free base form or in a different salt form. In some embodiments, the total daily dosage is about 1,500 to about 1,600 mg of Compound 1 in HCl salt form or an equivalent amount of Compound 1 in free base form or in a different salt form. In some embodiments, the total daily dosage is about 1,600 mg of Compound 1 in HCl salt form or an equivalent amount of Compound 1 in free base form or in a different salt form. In some embodiments, the total daily dosage is about 1,100 mg to about 1,300 mg of Compound 1 in HCl salt form or an equivalent amount of Compound 1 in free base form or in a different salt form. In some embodiments, the total daily dosage is about 1,236 mg of Compound 1 in HCl salt form or an equivalent amount of Compound 1 in free base form or in a different salt form.

In some embodiments, Compound 1 is administered once daily. In some embodiments, Compound 1 is administered twice daily.

Other objects, features, and advantages of the present invention will be apparent to one of skill in the art from the following detailed description and figures.

### BRIEF DESCRIPTION OF THE DRAWINGS

FIG. 1 plots the Western Blot percent stabilization for certain variants of TTR in the presence of Compound 1 (Acoramidis, column on left (10 μM) or Tafamidis (center (26 μM) and column on right (16 μM)). SD is only shown for conditions with two or more samples.

FIG. 2 shows a gel from the western blot assay of an individual V112I patient sample. All conditions were run in duplicate lanes. Brackets denoting the bands corresponding to tetrameric TTR with or without Retinol Binding Protein (RBP) are indicated.

FIG. 3 shows an FPE time course of an indivudal with a V122I TTR mutation. The doses tested were 10 μM Acoramidis, also referred to herein a Compound 1 (filled squares); 26 μM Tafamidis (filled circles); 16 μM Tafamidis (filled triangles); and DMSO (open circles).

FIG. 4 shows the TTR FPE percent stabilization by variant. For each variant, the column on the left is 10 μM Acoramidis, also referred to herein a Compound 1; the column in the middle is 26 μM Tafamidis; and the column on the right is 16 μM Tafamidis.

FIG. 5A-5D shows stabilization of unique variant patient samples measured by (5A) western blot (WB) and (5B) fluorescent probe exclusion (FPE), with wildtype (WT) results as reference. With reference to panel (5A), Acoramidis, also referred to herein a Compound 1, is the left column; DMSO is the right column. Overall stabilization across all tested variant samples measured by (5C) WB and (5D) FPE.

FIG. 6A-6E shows Phase 3 ATTRibute-CM results in patients receiving Compound 1 in a total daily dose of 1,600 mg (800 mg b.i.d.) or placebo. The reults demonstrate that ex vivo TTR stabilization correlates with in vivo measurement of serum TTR. (6A) shows mean change from baseline in serum TTR at month 30; (6B) shows median of WB % stabilization at month 30; (6C) shows median of FPE % stabilization at month 30; (6D) is a waterfall plot of variant patients WB % stabilization month 30; and (6E) is a waterfall plot of variant patients change from baseline in serum TTR at Month 30. Note: variant=mutant TTR genotype. Participants in ATTRibute-CM had the option for concomitant tafamidis use on top of blinded acoramidis or placebo in the study. For individuals on concomitant tafamidis, mean time on tafamidis in the study was 11 months.

### DETAILED DESCRIPTION OF THE INVENTION

### I. General

Described herein are methods for treating transthyretin (TTR) amyloidosis in a subject. Surprisingly, subjects with certain mutations in TTR responded surprisingly well to treatment.

### II. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Compound 1” refers to the chemical 3-(3-(3,5-dimethyl-1H-pyrazol-4-yl) propoxy)-4-fluorobenzoic acid (AG10, acoramidis), having the formula:

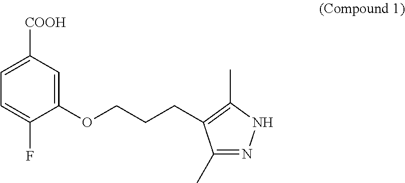 [SMILES: Cc1n[nH]c(C)c1CCCOc1cc(C(=O)O)ccc1F]

or a pharmaceutically acceptable salt thereof. When referring to specific amounts of Compound 1 administered to patients, this application refers to the amount of Compound 1 HCl salt administered. A person of skill in the art would recognize that in order to administer the same amount of Compound 1 in freebase or in a different salt form, small adjustments in overall amount administered is necessary. Compound 1 is described in International Patent Application No. PCT/US2013/076213, filed Dec. 18, 2013, which is herein incorporated by reference in its entirety. Crystalline and salt forms of Compound 1, as well as methods of making the same, are described in International Patent Application No. PCT/US2018/000025, filed Feb. 16, 2018, which is herein incorporated by reference in its entirety. Methods of treatment with Compound 1 are described in International Patent Application No. PCT/US2019/023555, filed Mar. 22, 2019, which is herein incorporated by reference in its entirety.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

As used herein, “treatment” or “treating,” or “palliating” or “ameliorating” are used interchangeably herein. These terms refer to an approach for obtaining beneficial or desired results including but not limited to a therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance. For example certain methods described herein treat transthyretin (TTR) amyloidosis by decreasing or reducing the occurrence, or progression of TTR fibril formation; or treat TTR amyloidosis by decreasing a symptom of TTR amyloidosis.

An “effective amount” or a “pharmaceutically effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control.

“Patient” or “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, and other non-mammalian animals. In some embodiments, a patient, subject, or subject in need thereof is a human.

### III. Detailed Description of the Embodiments

### Methods

In one aspect, provided herein is a method of treating transthyretin (TTR) amyloidosis. The method includes administering to a subject in need thereof a therapeutically effective amount of Compound 1, having the formula:

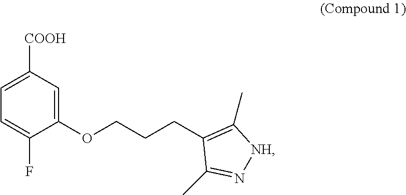 [SMILES: Cc1n[nH]c(C)c1CCCOc1cc(C(=O)O)ccc1F]

or a pharmaceutically acceptable salt thereof, where the therapeutically effective amount is a total daily dosage of about 10 milligrams (mg) to 2,000 mg. In some embodiments, the total daily dosage of Compound 1 is about 10 mg to about 50 mg, about 50 mg to about 300 mg, about 50 mg to about 150 mg, about 150 mg to about 800 mg, about 800 mg to about 1,600 mg, about 1,100 mg to about 1,300 mg, or about 800 mg to about 2,000 mg. In some embodiments, the total daily dosage of Compound 1 is about 10 mg to about 50 mg. In some embodiments, the total daily dosage of Compound 1 is about 50 mg to about 300 mg. In some embodiments, the total daily dosage of Compound 1 is about 50 mg to about 150 mg. In some embodiments, the total daily dosage of Compound 1 is about 150 mg to about 800 mg. In some embodiments, the total daily dosage of Compound 1 is about 800 mg to about 1,600 mg. In some embodiments, the total daily dosage of Compound 1 is about 1,200 mg to about 1,600 mg. In some embodiments, the total daily dosage of Compound 1 is about 800 mg to 2,000 mg. In some embodiments, the total daily dosage of Compound 1 is about 1,100 mg to about 1,300 mg. In some embodiments, the total daily dosage of Compound 1 is about 800 mg. In some embodiments, the total daily dosage of Compound 1 is about 1,600 mg. In some embodiments, the total daily dosage of Compound 1 is about 1,236 mg. It is understood that in the present disclosure the amount of Compound 1 listed is the amount of HCl salt of Compound 1 administered. A person of skill in the art would recognize that in order to administer the same amount of Compound 1 in freebase or in a different salt form, small adjustments in overall amount administered is necessary.

In some embodiments the total daily dosage of Compound 1 is about 10 mg. In some embodiments the total daily dosage of Compound 1 is about 25 mg. In some embodiments the total daily dosage of Compound 1 is about 50 mg. In some embodiments the total daily dosage of Compound 1 is about 1000 mg. In some embodiments the total daily dosage of Compound 1 is about 150 mg. In some embodiments the total daily dosage of Compound 1 is about 200 mg. In some embodiments the total daily dosage of Compound 1 is about 300 mg. In some embodiments the total daily dosage of Compound 1 is about 600 mg. In some embodiments the total daily dosage of Compound 1 is about 800 mg. In some embodiments the total daily dosage of Compound 1 is about 1,600 mg. In some embodiments the total daily dosage of Compound 1 is about 1,236 mg.

Compound 1 can be administered once (SID or qd), twice (BID or q12h), three (TID), or four times (QID) a day. In some embodiments, Compound 1 is administered once daily. In some embodiments, Compound 1 is administered twice daily. In some embodiments, Compound 1 is administered three times daily. In some embodiments, Compound 1 is administered four times daily.

In some embodiments, about 50 mg of Compound 1 is administered once daily. In some embodiments, about 150 mg of Compound 1 is administered once daily. In some embodiments, about 300 mg of Compound 1 is administered once daily. In some embodiments, about 800 mg of Compound 1 is administered once daily. In some embodiments, about 1,236 mg of Compound 1 is administered once daily.

In some embodiments, about 100 mg of Compound 1 is administered twice daily. In some embodiments, about 300 mg of Compound 1 is administered twice daily. In some embodiments, about 400 mg of Compound 1 is administered twice daily. In some embodiments, about 800 mg of Compound 1 is administered twice daily.

In another aspect, provided herein are methods of treating transthyretin (TTR) amyloidosis. The methods include administering to a subject in need thereof a therapeutically effective amount of Compound 1, having the formula:

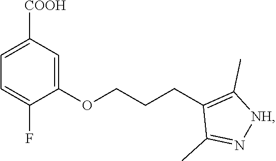 [SMILES: Cc1n[nH]c(C)c1CCCOc1cc(C(=O)O)ccc1F]

or a pharmaceutically acceptable salt thereof, where the therapeutically effective amount of Compound 1 maintains a trough steady-state blood plasma concentration of Compound 1 of at least about 5 micromolar (μM). In some embodiments, the therapeutically effective amount of Compound 1 maintains a trough steady-state blood plasma concentration of Compound 1 of at least about 6 μM. In some embodiments, the therapeutically effective amount of Compound 1 maintains a trough steady-state blood plasma concentration of Compound 1 of at least about 6.5 μM. In some embodiments, the therapeutically effective amount of Compound 1 maintains a trough steady-state blood plasma concentration of Compound 1 of at least about 7 μM. In some embodiments, the therapeutically effective amount of Compound 1 maintains a trough steady-state blood plasma concentration of Compound 1 of at least about 7.5 μM. In some embodiments, the therapeutically effective amount of Compound 1 maintains a trough steady-state blood plasma concentration of Compound 1 of at least about 8 μM.

In some embodiments, the therapeutically effective amount of Compound 1 maintains a trough steady-state blood plasma concentration of Compound 1 from about 5 to about 30 μM. In some embodiments, the therapeutically effective amount of Compound 1 maintains a trough steady-state blood plasma concentration of Compound 1 from about 5 to about 25 μM. In some embodiments, the therapeutically effective amount of Compound 1 maintains a trough steady-state blood plasma concentration of Compound 1 from about 6 to about 20 μM. In some embodiments, the therapeutically effective amount of Compound 1 maintains a trough steady-state blood plasma concentration of Compound 1 from about 7.5 to about 15 μM. In some embodiments, the therapeutically effective amount of Compound 1 maintains a trough steady-state blood plasma concentration of Compound 1 from about 7.5 to about 10 μM. In some embodiments, the therapeutically effective amount of Compound 1 maintains a trough steady-state blood plasma concentration of Compound 1 from about 8 to about 10 μM.

In some embodiments, subjects receiving a therapeutically effective amount of Compound 1, or a pharmaceutically acceptable salt thereof experience an increase in transthyretin (TTR) blood serum concentrations relative to baseline levels. In some embodiments, subjects receiving a therapeutically effective amount of Compound 1, or a pharmaceutically acceptable salt thereof experience at least about a 10, 15, 20, 25, 30% or more increase in transthyretin (TTR) blood serum concentrations relative to baseline levels after 28 days of treatment. In some embodiments, subjects receiving a therapeutically effective amount of Compound 1, or a pharmaceutically acceptable salt thereof experience at least about a 25% increase in transthyretin (TTR) blood serum concentrations relative to baseline levels after 28 days of treatment. In some embodiments, subject prior to treatment have TTR blood serum levels that are beneath a baseline serum TTR concentration (20 mg/dL TTR). In some embodiments, subject receiving an effective amount of Compound 1, or a pharmaceutically acceptable salt thereof for 28 days will experience increased blood serum TTR levels such that the level of blood serum TTR is above the baseline level. In some embodimnets, the subject experiencing increase TTR level are subjects diagnosed with transthyretin amyloidosis (ATTR) cardiomyopathy.

In some embodiments, the TTR amyloidosis in the subject is characterized by a TTR protein comprising a mutation selected from the group consisting of
a glycine to serine mutation at position 6 (G6S),
an alanine to serine mutation at position 25 (A25S),
a valine to methionine mutation at position 30 (V30M)
an alanine to aspartic acid mutation at position 36 (A36D),
a glutamic acid to aspartic acid mutation at position 42 (E42D),
a serine to arginine mutation at position 50 (S50R),
a threonine to alanine mutation at position 60 (T60A)
an isoleucine to leucine mutation at position 68 (I68L),
a glutamic acid to glutamine mutation at position 89 (E89Q),
a glutamic acid to glutamine mutation at position 92 (E92Q),
a valine to leucine mutation at position 94 (V94L),
a valine to isoleucine mutation at position 122 (V122I),
an alanine to serine mutation at position 97 (A97S),
an aspartic acid to alanine mutation at position 38 (D38A),
a phenylalanine to leucine mutation at position 64 (F64L),
a leucine to histidine mutation at position 58 (L58H),
a proline to serine mutation at position 24 (P24S), and
a tyrosine to cysteine mutation at position 114 (Y114C). The wild-type transthyretin (TTR) protein is provided herein as SEQ ID NO: 1.

In some embodiments, the TTR amyloidosis in the subject is characterized by a TTR protein comprising a mutation selected from the group consisting of a glycine to serine mutation at position 6 (G6S),
an alanine to serine mutation at position 25 (A25S),
an alanine to aspartic acid mutation at position 36 (A36D),
a glutamic acid to aspartic acid mutation at position 42 (E42D),
a serine to arginine mutation at position 50 (S50R),
an isoleucine to leucine mutation at position 68 (168L),
a glutamic acid to glutamine mutation at position 89 (E89Q),
a glutamic acid to glutamine mutation at position 92 (E92Q),
a valine to leucine mutation at position 94 (V94L), and
an alanine to serine mutation at position 97 (A97S). The wild-type transthyretin (TTR) protein is provided herein as SEQ ID NO: 1.

In some embodiments, the TTR amyloidosis in said subject is characterized by a TTR protein comprising a glycine to serine mutation at position 6 (G6S).

In some embodiments, the TTR amyloidosis in said subject is characterized by a TTR protein comprising an alanine to serine mutation at position 25 (A25S).

In some embodiments, the TTR amyloidosis in said subject is characterized by a TTR protein comprising a valine to methionine mutation at position 30 (V30M).

In some embodiments, the TTR amyloidosis in said subject is characterized by a TTR protein comprising an alanine to aspartic acid mutation at position 36 (A36D).

In some embodiments, the TTR amyloidosis in said subject is characterized by a TTR protein comprising a glutamic acid to aspartic acid mutation at position 42 (E42D).

In some embodiments, the TTR amyloidosis in said subject is characterized by a TTR protein comprising a serine to arginine mutation at position 50 (S50R).

In some embodiments, the TTR amyloidosis in said subject is characterized by a TTR protein comprising a threonine to alanine mutation at position 60 (T60A).

In some embodiments, the TTR amyloidosis in said subject is characterized by a TTR protein comprising an isoleucine to leucine mutation at position 68 (168L).

In some embodiments, the TTR amyloidosis in said subject is characterized by a TTR protein comprising a glutamic acid to glutamine mutation at position 89 (E89Q).

In some embodiments, the TTR amyloidosis in said subject is characterized by a TTR protein comprising a glutamic acid to glutamine mutation at position 92 (E92Q).

In some embodiments, the TTR amyloidosis in said subject is characterized by a TTR protein comprising a valine to leucine mutation at position 94 (V94L).

In some embodiments, the TTR amyloidosis in said subject is characterized by a TTR protein comprising a valine to isoleucine mutation at position 122 (V122I).

In some embodiments, the TTR amyloidosis in said subject is characterized by a TTR protein comprising an alanine to serine mutation at position 97 (A97S).

In some embodiments, the TTR amyloidosis in said subject is characterized by a TTR protein comprising an aspartic acid to alanine mutation at position 38 (D38A).

In some embodiments, the TTR amyloidosis in said subject is characterized by a TTR protein comprising a phenylalanine to leucine mutation at position 64 (F64L).

In some embodiments, the TTR amyloidosis in said subject is characterized by a TTR protein comprising a leucine to histidine mutation at position 58 (L58H).

In some embodiments, the TTR amyloidosis in said subject is characterized by a TTR protein comprising a proline to serine mutation at position 24 (P24S).

In some embodiments, the TTR amyloidosis in said subject is characterized by a TTR protein comprising a tyrosine to cysteine mutation at position 114 (Y114C).

There are a variety of diseases or disorders associated with transthyretin (TTR) amyloidosis. These include, but are not limited to familial amyloid polyneuropathy, familial amyloid cardiomyopathy, senile systemic amyloidosis, central amyloidosis, ocular amyloidosis, Leptomeningeal amyloidosis, oculoleptomeningeal amyloidosis, vitreous amyloidosis, gastrointestinal amyloidosis, neuropathic amyloidosis, non-neuropathic amyloidosis, non-hereditary amyloidosis, reactive/secondary amyloidosis, cerebral amyloidosis.

In some embodiments, the transthyretin (TTR) amyloidosis disease is transthyretin amyloidosis (ATTR) cardiomyopathy. In some embodiments, the transthyretin (TTR) amyloidosis disease is transthyretin amyloidosis (ATTR) polyneuropathy.

ATTR cardiomyopathy includes wild-type ATTR cardiomyopathy (ATTRwt-CM) and genetic (familial) ATTR cardiomyopathy (ATTRm-CM). ATTRm-CM is caused by a mutation in the TTR protein, whereas ATTRwt-CM is not caused by a mutation. Instead, ATTRwt-CM is generally an age-related process. In some embodiments, the ATTR cardiomyopathy is ATTRwt-CM. In some embodiments, the ATTR cardiomyopathy is ATTRm-CM.

ATTR polyneuropathy includes both wild type & genetic (familial) ATTR polyneuropathy. As discussed for cardiomyopathy, ATTRm-PN is caused by a mutation in the TTR protein, whereas ATTRwt-PN does not include a genetic component. In some embodiments ATTR-PN is ATTRwt-PN. In some embodiments, ATTR-PN is ATTRm-PN.

ATTR cardiomyopathy (both wild-type and familial) is a slowly progressive disease that causes heart failure and death in affected subjects. The methods of the present disclosure, provide clinical improvement in subjects with ATTR cardiomyopathy by stopping or slowing the accumulation of TTR fibrils in the myocardium. Through this process, the currently described methods provide clinical improvements in ATTR cardiomyopathy subjects. Clinical improvements include, but are not limited to, improvement in New York Heart Association (NYHA) functional classification, improvements in Kansas City Cardiomyopathy Questionnaire responses, EuroQoL-5 Dimensions (EQ-5D-5L), improvement in 6 minute walk test performance, improvement in markers associated with cardiac health such as Troponin T, Troponin I, B-type natriuretic peptide (BNP), and N-terminal pro-BNP, decreasing the frequency of cardiovascular-related hospitalizations, and/or decreasing mortality.

In some embodiments, the methods provided herein improve, stabilize or delay worsening in New York Heart Association (NYHA) functional classification of subjects. The NYHA functional classification grades the severity of heart failure symptoms as one of four functional classes. The NYHA functional classification is widely used in clinical practice and in research because it provides a standard description of severity that can be used to assess response to treatment and to guide management. The NYHA functional classification is based on severity of symptoms and limitation of physical activity:
Class I: No limitation of physical activity. Ordinary physical activity does not cause undue breathlessness, fatigue, or palpitations.
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

In some embodiments, administration of a therapeutically effective amount of Compound 1, or a pharmaceutically acceptable salt thereof reduces the New York Heart Association (NYHA) functional classification of the subject. In some embodiments, administration of a therapeutically effective amount of Compound 1, or a pharmaceutically acceptable salt thereof, in a subject with ATTR cardiomyopathy improves, stabilizes or delays worsening in New York Heart Association (NYHA) functional classification of subjects. In some embodiments, administration of a therapeutically effective amount of Compound 1, or a pharmaceutically acceptable salt thereof, reduces the NYHA functional classification of the subject. In some embodiments, the NYHA functional classification is reduced from class IV to class III, from class IV to class II, or from class IV to class I. In some embodiments, the NYHA functional classification is reduced from class IV to class III. In some embodiments, the NYHA functional classification is reduced from class IV to class II. In some embodiments, the NYHA functional classification is reduced from class III to class II. In some embodiments, the NYHA functional classification is reduced from class III to class I. In some embodiments, the NYHA functional classification is reduced from class II to class I.

In some embodiments, the methods provided herein improve, stabilize or delay worsening in Kansas City Cardiomyopathy Questionnaire (KCCQ) classification of subjects. In some embodiments, the methods described herein provide improved scores in the Kansas City Cardiomyopathy Questionnaire (KCCQ) (Green C P, et al. (2000) Journal of the American College of Cardiology 35:1245-55), the contents of which are incorporated herein by reference for all purposes. The KCCQ contains specific questions related to cardiac health and provides valid, reliable and sensitive measures of disease-specific health-related quality of life.

The questions of the KCCQ ask subjects to assess how limited (e.g. severely limited, limited quite a bit, moderately limited, slightly limited, or did not limit at all) they are in performing normal aspects of their life. In some embodiments, subjects have an average improvement of at least one level (e.g. severely limited to limited quite a bit, limited quite a bit to moderately limited, moderately limited to slightly limited) on all questions of the questionnaire after treatment with Compound 1, or a pharmaceutically acceptable salt thereof.

In some embodiments, the methods provided herein improve, stabilize or delay worsening in EuroQoL-5 Dimensions (EQ-5D-5L) score in subjects. EQ-5D-5L is a brief, self-administered generic health status instrument that takes about 5 minutes to complete. The instrument includes two parts. In the first part, respondents are asked to rate their current health state on 5 dimensions (mobility, self-care, usual activities, pain or discomfort, and anxiety or depression) with each dimension having five levels of function (1-no problem, 2-slight problem, 3-moderate problem, 4-severe problem, and 5-extreme problem). The second part is a respondents self-rating of current health status on a Visual Analog Scale (EQ VAS) with endpoints labeled “best imaginable health state” (score of 100) and “worst imaginable health state” (score of 0). The scores from the 5 dimensions may be used to calculate a single index value, also known as a utility score. The EQ-5D-5L questionnaire is in the public domain and can be obtained from EuroQoL.

In some embodiments, patients receiving the treatment methods described herein have an average improvement of at least one, tow, three four, five, six, seven, either, nine, or ten points in the EuroQoL-5 Dimensions (EQ-5D-5L) utility score. In some embodiments, patients receiving the treatment methods described herein have an average improvement of at least five points in the EuroQOL-5 Dimensions (EQ-5D-5L) utility score.

In some emboimdnets, administration of a therapeutically effective amount of Compound 1, or a pharmaceutically acceptable salt thereof in a subject with ATTR cardiomyopathy improves the subject's risk of mortality, morbidity, and quality of life.

In some embodiments, the methods described herein improve a subject's six minute walk test performance. The six minute walk test (6MWT) is a self-paced timed walk for six minutes to assess the level of functional capacity of a subject. Subjects are allowed to stop and rest during the test if the levels of exertion exceed their comfort level. Assessment before, after, and during treatment is relatively easy to assess and consists of measuring the distance the subject walks in a six-minute time period. Thus, in some embodiments, subjects increase total distance covered in the six minute walking test after treatment with Compound 1, or a pharmaceutically acceptable salt thereof. In some embodiments, subjects walked at least 25 m further than a baseline distance measured prior to treatment with Compound 1, or a pharmaceutically acceptable salt thereof. In some embodiments, subjects walked at least 30 m further than a baseline distance measured prior to treatment with Compound 1, or a pharmaceutically acceptable salt thereof. In some embodiments, subjects walked at least 50 m further than a baseline distance measured prior to treatment with Compound 1, or a pharmaceutically acceptable salt thereof. In some embodiments, subjects walked at least 75 m further than a baseline distance measured prior to treatment with Compound 1, or a pharmaceutically acceptable salt thereof. In some embodiments, subjects walked at least 100 m further than a baseline distance measured prior to treatment with Compound 1, or a pharmaceutically acceptable salt thereof. In some embodiments, subjects receiving the treatment methods described herein have a slowed reduction in six-minute walking distance. For example, in some embodiments, a subject mantains about the same six minute walking distance as prior to treatment. In some embodiments, a subject covers 10 m less ina a six minute wakling test. In some embodimnets, the six minute walk test is used to compare the treatment group to the non-treatment group. In some embodiments, the between group mean change from baseline is at least 10 meters. In some embodiments, the between group mean change from baseline is at least 20 meters. In some embodiments, the between group mean change from baseline is at least 30 meters. In some embodiments, the treatment methods provided herein reduced the decline in the six minute walk test distance as compared to individuals not receiving treatment.

In some embodiments, administration of a therapeutically effective amount of Compound 1, or a pharmaceutically acceptable salt thereof, in a subject with ATTR cardiomyopathy decreases mortality as compared to subjects not receiving treatment.

In some embodiments, administration of a therapeutically effective amount of Compound 1, or a pharmaceutically acceptable salt thereof, in a subject with ATTR cardiomyopathy decreases all cause mortality as compared to subjects not receiving treatment.

Troponin T, Troponin I, Brain natriuretic peptide (BNP), and N-terminal pro-BNP are polypeptides that are elevated in serum blood of subjects with poor myocardial health. In some embodiments, the level of Troponin T, Troponin I, BNP and/or N-terminal pro-BNP decrease after treatment with Compound 1, or a pharmaceutically acceptable salt thereof. In some embodiments, the level of Troponin T, Troponin I, BNP and/or N-terminal pro-BNP decrease about 10% as compared to a baseline level of Troponin T, Troponin I, BNP and/or N-terminal pro-BNP in said subject prior to treatment with Compound 1, or a pharmaceutically acceptable salt thereof. In some embodiments, the level of Troponin T, Troponin I, BNP and/or N-terminal pro-BNP decrease about 15% as compared to a baseline level of Troponin T, Troponin I, BNP and/or N-terminal pro-BNP in said subject prior to treatment with Compound 1, or a pharmaceutically acceptable salt thereof.

In some embodiments, administration of a therapeutically effective amount of Compound 1, or a pharmaceutically acceptable salt thereof, in a subject with ATTR cardiomyopathy decreases serum blood levels of brain natriuretic peptide (BNP).

In some embodiments, administration of a therapeutically effective amount of Compound 1, or a pharmaceutically acceptable salt thereof, in a subject with ATTR cardiomyopathy decreases serum blood levels of N-terminal pro-brain natriuretic peptide (N-terminal pro-BNP).

As discussed above, a clinical improvement provided in some embodiments of the disclosed methods, is decreasing the rate of cardiovascular related hospitalizations in subjects receiving treatment as compared to those who do not receive treatment. In some embodiments, patents on average at least 0.5, 1, 1.5, 2, 3, 4, 5 fewer cardiovascular related hospitalizations per year as compared to those who do not receive treatment.

An additional clinical benefit that is provided in some embodiments of the methods disclosed herein, is a decrease in mortality rate as compared to individuals not receiving treatment. In some embodiments, mortality rate is reduced by about 2, 4, 6, 8, 10, 12, 14, 16, 18, 20, 22, 24, 26, 28, 30 or more percent as compared to subjects who did not receive treatment.

ATTR polyneuropathy is a disease where TTR amyloid (ATTR) deposits impair or otherwise compromise normal nerve function. ATTR polyneuropathy is a progressive disease that causes cachexia and death in affected subjects. The methods of the present disclosure, provide clinical improvement in subjects with ATTR polyneuropathy by stopping or slowing the accumulation of TTR fibrils. Through this process, the currently described methods provide clinical improvements ATTR polyneuropathy subjects. Clinical improvements include, but are not limited to, improvements in Neuropathy Impairment Score (NIS) or modified Neuropathy Impairment Score+7 (mNIS+7), improvements in Norfolk Quality of Life Diabetic Neuropathy questionnaire, improvements in composite autonomic symptom score (COMPASS-31) score, improved nutritional status as measured by modified body mass index (mBMI), and/or improvements in a subject's 10 meter walk test.

In some embodiments, the methods described herein provide improved Neuropathy Impairment Score (NIS). NIS refers to a scoring system that measures weakness, sensation, and reflexes. The NIS score evaluates a standard group of muscles for weakness (1 is 25% weak, 2 is 50% weak, 3 is 75% weak, 3.25 is movement against gravity, 3.5 is movement with gravity eliminated, 3.75 is muscle flicker without movement, and 4 is paralyzed), a standard group of muscle stretch reflexes (0 is normal, 1 is decreased, 2 is absent), and touch-pressure, vibration, joint position and motion, and pinprick (all graded on index finger and big toe: 0 is normal, 1 is decreased, 2 is absent). Evaluations are corrected for age, gender, and physical fitness.

In some embodiments, the methods described herein slow the progression of the disease such that the rate of NIS score increase is reduced as compared to a subject who is not taking Compound 1, or a pharmaceutically acceptable salt thereof. In some embodiments, the methods described herein stop the progression of the disease such that there is no change in NIS score after treatment with Compound 1, or a pharmaceutically acceptable salt thereof.

In some embodiments, the methods described herein reduce the NIS score after treatment with Compound 1, or a pharmaceutically acceptable salt thereof. In some embodiments, the methods described herein reduced the NIS score by at least 5, 10, 15, 20, 25, 30, 35, 40, 45, 50, 55, or 60% as compared to a baseline level measured prior to treatment with Compound 1, or a pharmaceutically acceptable salt thereof. In some embodiments, the methods described herein reduced the NIS score by at least 5% as compared to a baseline level measured prior to treatment with Compound 1, or a pharmaceutically acceptable salt thereof. In some embodiments, the methods described herein reduced the NIS score by at least 10% as compared to a baseline level measured prior to treatment with Compound 1, or a pharmaceutically acceptable salt thereof. In some embodiments, the methods described herein reduced the NIS score by at least 15% as compared to a baseline level measured prior to treatment with Compound 1, or a pharmaceutically acceptable salt thereof.

In some embodiments, the methods described herein provide improved modified Neuropathy Impairment Score (mNIS+7). mNIS+7 refers to a clinical exam-based assessment of neurologic impairment (NIS) combined with electrophysiologic measures of small and large nerve fiber function (NCS and QST), and measurement of autonomic function (postural blood pressure). The mNIS+7 score is a modification of the NIS+7 score (which represents NIS plus seven tests). NIS+7 analyzes weakness and muscle stretch reflexes. Five of the seven tests include attributes of nerve conduction. These attributes are the peroneal nerve compound muscle action potential amplitude, motor nerve conduction velocity and motor nerve distal latency (MNDL), tibial MNDL, and sural sensory nerve action potential amplitudes. These values are corrected for variables of age, gender, height, and weight. The remaining two of the seven tests include vibratory detection threshold and heart rate decrease with deep breathing. The mNIS+7 score modifies NIS+7 to take into account the use of Smart Somatotopic Quantitative Sensation Testing, new autonomic assessments, and the use of compound muscle action potential of amplitudes of the ulnar, peroneal, and tibial nerves, and sensory nerve action potentials of the ulnar and sural nerves (Suanprasert, N. et al., Retrospective study of a TTR FAP cohort to modify NIS+7 for therapeutic trials, J. Neurol. Sci., 2014. 344 (1-2): pgs. 121-128). Further details of the mNIS+7 exam can be found in US 2017/0307608, the contents of which is incorporated herein by reference, for all purposes.

In some embodiments, the methods described herein slow the progression of the disease such that the rate of mNIS+7 score increase is reduced as compared to a subject who is not taking Compound 1, or a pharmaceutically acceptable salt thereof. In some embodiments, the methods described herein stop the progression of the disease such that there is no change in mNIS+7 score after treatment with Compound 1, or a pharmaceutically acceptable salt thereof.

In some embodiments, the methods described herein reduce the mNIS+7score after treatment with Compound 1, or a pharmaceutically acceptable salt thereof. In some embodiments, the methods described herein improves the modified Neuropathy Impairment Score+7 (mNIS+7) in said subject. In some embodiments, the methods described herein reduced the mNIS+7score by at least 5, 10, 15, 20, 25, 30, 35, 40, 45, 50, 55, or 60% as compared to a baseline level measured prior to treatment with Compound 1, or a pharmaceutically acceptable salt thereof. In some embodiments, the methods described herein reduced the mNIS+7 score by at least 5% as compared to a baseline level measured prior to treatment with Compound 1, or a pharmaceutically acceptable salt thereof. In some embodiments, the methods described herein reduced the mNIS+7 score by at least 10% as compared to a baseline level measured prior to treatment with Compound 1, or a pharmaceutically acceptable salt thereof. In some embodiments, the methods described herein reduced the mNIS+7 score by at least 15% as compared to a baseline level measured prior to treatment with Compound 1, or a pharmaceutically acceptable salt thereof.

In some embodiments, the methods described herein provide improved scores in the Norfolk Quality of Life Diabetic Neuropathy (QOL-DN) questionnaire. This questionnaire is well known to a person of skill in the art, and is a validated questionnaire that captures pain related to large fiber, small fiber, and autonomic neuropathy. The questionnaire includes items related to symptoms experienced by the subject and questions related to the impact of neuropathy on the daily activities of a subject.

In some embodiments, the methods described herein slow the progression of the disease such that the rate of the Norfolk QOL-DN score decline is reduced as compared to a subject who is not taking Compound 1, or a pharmaceutically acceptable salt thereof. In some embodiments, the methods described herein stop the progression of the disease such that there is no change in the Norfolk QOL-DN score after treatment with Compound 1, or a pharmaceutically acceptable salt thereof.

In some embodiments, the methods described herein slow the progression of the disease such that there is no change in the Norfolk QOL-DN score after treatment with Compound 1, or a pharmaceutically acceptable salt thereof.

In some embodiments, the methods described herein improve the Norfolk QOL-DN score after treatment with Compound 1, or a pharmaceutically acceptable salt thereof. In some embodiments, the methods described herein improve the Norfolk QOL-DN by at least 5, 10, 15, 20, 25, 30, 35, 40, 45, 50, 55, or 60% as compared to a baseline level measured prior to treatment with Compound 1, or a pharmaceutically acceptable salt thereof. In some embodiments, the methods described herein improve the Norfolk QOL-DN score by at least 5% as compared to a baseline level measured prior to treatment with Compound 1, or a pharmaceutically acceptable salt thereof. In some embodiments, the methods described herein improve the Norfolk QOL-DN score by at least 10% as compared to a baseline level measured prior to treatment with Compound 1, or a pharmaceutically acceptable salt thereof. In some embodiments, the methods described herein improve the Norfolk QOL-DN score by at least 15% as compared to a baseline level measured prior to treatment with Compound 1, or a pharmaceutically acceptable salt thereof. In some embodiments, the methods described herein provide a change in a subject's Norfolk QOL-DN of about −1.5, −2.0, −2.5, −3.0, −3.5, −4.0, −4.5, −5.0, −5.5, −6.0, −6.7, −7.0, −7.5, −8.0, −8.5, −9.0, −9.5, or −10.0 as compared to a subject's baseline score.

In some embodiments, the methods disclosed herein provide imporved composite autonomic symptom score (COMPASS-31). The composite autonomic symptom score (COMPASS-31) is a patient questionnaire that assesses symptoms of dysautonomia. In one embodiment, the methods of the invention provide to the subject an improvement versus baseline in a COMPASS-31 score. Such an improvement can take the form of an increase of at least 0.1, for example at least 0.2, at least 0.3, at least 0.4, or at least 0.5, e.g., 0.1, 0.2, 0.3, 0.4, or 0.5, points of the subject's COMPASS-31 score. In some embodiments, the methods slow the progression of the disease such that there is no change in the COMPASS-31 score. In yet other embodiments, the methods of the invention slow the rate at which a COMPASS-31 score decreases, e.g., the rate of decrease of a COMPASS-31 score in a subject treated with AG10 as compared to the rate of decrease of a COMPASS-31 score in a subject that is not treated with AG10.

In some embodiments, the methods disclosed herein provide imporved nutritional status as measured by modified body mass index (mBMI), which is determined by multiplying the BMI of an individuals by their serum albumin levels. The calculation of mBMI accounts for the contribution of edema to total weight. In one embodiment, the methods of the disclosure provide to the subject an improvement versus baseline in mBMI. Such an improvement can take the form of a mBMI score decrease of about 2, 5, 7, 10, 12, 15, 20, or about 25. In other embodiments, the methods arrest an increasing mBMI index score, e.g., the methods result in a 0% increase of the mBMI score. In yet other embodiments, the methods of the invention slow the rate at which mBMI score increases, e.g., the rate of increase of a mBMI score in a subject treated with AG10 as compared to the rate of increase of a mBMI score in a subject that is not treated with AG10.

In some embodiments, the methods disclosed herein provide improvements in the 10-meter walk test (10MWT). This test measures an individuals walking speed over 10 meters. In one embodiment, the methods of the disclosure provide to the subject an increase from baseline in the 10-meter walk test. In some embodiments, the increase from baseline in the 10-meter walk test is about 0.025, 0.03, 0.04, 0.05, 0.06, 0.07, 0.08, 0.09, 1.0, 1.5, 2.0, 2.5, 3.0, 3.5, 4.0. 4.5, or about 5.0 meters/second.

In some embodiments, the methods disclosed herein provide imporved Dyck/Rankin scores. The Dyck/Rankin score is known in the art, and is assigned by a physician after assessing the patient's symptoms, neuropathic impairments, test results, and reviewing a patient's ability to perform acts of daily living. Only disability related to peripheral neuropathy is graded. In deciding whether the patient has difficulty or inability to perform certain tasks or acts of daily living, more than the patient's report should be used for judgment; the physician should use objective criteria. The stages (0-8) are outlined below:
0. No neuropathy
   No symptoms (NSS<1), signs (NIS<2 points); or tests (e.g. 7 tests<97.5th) abnormalities of neuropathy.
1. Minimal neuropathy (only one of A, B. C are abnormal)
   a) Tests, are the only abnormality (e.g. 7 test>97.5th); or
   b) Neuropathy signs, are the only abnormality (e.g. NIS>2 points); or
   c) Neuropathy symptoms are the only abnormality (e.g. NSS>1)
2. Minimal neuropathy: la+1b.
3. Symptomatic neuropathy: la, la+1c; la+1c or 1b+1c. The patient is able to continue with usual-acts of daily living, work or recreational activity, and can meet usual-family and social responsibilities.
   Symptoms of neuropathy: NSS≥1 symptoms of muscle weakness, atrophy or cramps; negative or positive, neuropathic sensory symptoms (N-NNS,P-NSS); or neuropathic autonomic symptoms.
   Usual acts of daily living, work, recreational and social and family activities: Despite neuropathic symptoms, the patient is able to work at his usual activity, maintain his usual home obligations and participate in recreational activities. Generally, the patient can carry-on despite some motor, sensory or autonomic symptoms. The patients may be unable to perform extraordinary activities e.g. competitive sports, feats of endurance, etc.
4. Symptomatic neuropathy (defined in 3) interfering and limiting work, usual acts of daily living, recreational activity or family and social obligations but independent function is possible without the help of others. At this score, there is an unequivocal limitation of usual* work, acts of living or recreational, family or social obligations * because of neuropathy.
   The degree of motor, sensory or autonomic symptoms or impairment is of a sufficient degree to limit the ability to work, to perform usual acts of daily living, recreational activities or to meet family and usual social responsibilities. The use of a can or orthotic device probably places the patient in this (or a higher) category unless the patient is able to perform “usual” acts of daily living, recreational activities and meet social and family responsibilities (then they would fall into lower category).
5. Symptomatic neuropathy (defined in 3) restricting acts of daily living, work and recreational activities. The help of other care-givers* (<2½ hrs/day) is needed. If use of a wheelchair is mandatory for acts of daily loving, recreational activities or social and family responsibilities, the patient would usually fall into this or a higher score (>5).
   A member of the family or visiting nurse is needed to provide acts of daily living (bathing, shaving, tooth brushing, feeding, etc.), daily management of analgesics or opiates or help with management of autonomic dysfunction which the patient is not able to perform adequately or safely on their own.
6 Symptomatic neuropathy (defended in 3) requiring the help of care-givers≥2½ hrs to <8 hrs/day as described in 5.
7. Symptomatic neuropathy (defined in 3) requiring the help of care giver>8 hrs/day but not continuously as in stage 8.
8. Symptomatic neuropathy (defined in 3) requiring constant care in an intensive care unit.

The time period for administration will depend on a number of factors including the specific disease being treated. For example, in particular transthyretin (TTR) amyloidosis diseases or condition, there is a genetic component such that chronic (i.e. continuous, long term) administration may be required. However, in some embodiments, administration of Compound 1, or a pharmaceutically acceptable salt thereof to subject with a genetic TTR amyloidosis disease will continue while the subject is demonstrating or experiencing symptoms related to the TTR amyloidosis disease or condition, or for a set period of time after a particular end point is met (e.g. reduction or complete elimination of symptoms). If the symptoms of the TTR amyloidosis disease return or begin to reappear, administration of Compound 1, or a pharmaceutically acceptable salt thereof is re-started.

For subjects with a non-genetic linked TTR amyloidosis disease, a number administration options are available and will depend on the severity of the disease and the clinical symptoms presented. In some embodiments, long term administration of Compound 1, or a pharmaceutically acceptable salt thereof is necessary. In some embodiments, shorter term, or acute, administration of Compound 1, or a pharmaceutically acceptable salt thereof is necessary. In some embodiments, administration of Compound 1, or a pharmaceutically acceptable salt thereof to a subject with a non-genetic linked TTR amyloidosis disease is continued while the subject is demonstrating or experiencing symptoms related to the TTR amyloidosis disease or condition, or for a set period of time after a particular end point is met (e.g. reduction or complete elimination of symptoms). If the symptoms of the TTR amyloidosis disease return or begin to reappear, administration of Compound 1, or a pharmaceutically acceptable salt thereof is re-started.

In some embodiments, Compound 1, or a pharmaceutically acceptable salt thereof is administered for at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, 100 days or longer. In some embodiments, Compound 1, or a pharmaceutically acceptable salt thereof is administered for 7, 14, 21, 28, 35, 42, 49, or 56 days. In some embodiments, Compound 1, or a pharmaceutically acceptable salt thereof is administered for 28 days. In some embodiments, Compound 1, or a pharmaceutically acceptable salt thereof is administered for 56 days. In some embodiments, Compound 1, or a pharmaceutically acceptable salt thereof is administered for 84 days.

In some embodiments, Compound 1, or a pharmaceutically acceptable salt thereof is administered for at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 38, 39, 40, 41, 42, 43, 44, 45, 46, 47, 48, 49, 50 months or. In some embodiments, Compound 1, or a pharmaceutically acceptable salt thereof is administered for 10, 15, 20, 25, 30, 35, 40, 45, or 50 months. In some embodiments, Compound 1, or a pharmaceutically acceptable salt thereof is administered for 6 months. In some embodiments, Compound 1, or a pharmaceutically acceptable salt thereof is administered for 12 months. In some embodiments, Compound 1, or a pharmaceutically acceptable salt thereof is administered for 18 months. In some embodiments, Compound 1, or a pharmaceutically acceptable salt thereof is administered for 24 months. In some embodiments, Compound 1, or a pharmaceutically acceptable salt thereof is administered for 30 months. In some embodiments, Compound 1, or a pharmaceutically acceptable salt thereof is administered for 36 months. In some embodiments, Compound 1, or a pharmaceutically acceptable salt thereof is administered for 42 months.

Advantageously, agents used in diuretic therapy did not alter the exposure of AG10 during treatment. As such, patients receiving diuretic therapy can be administered AG10 without altered or specialized dosing regimens. Thus, in some embodiments, the subject receiving AG10 is also receiving an additional diuretic therapy agent. Diuretic therapy agents include, but are not limited to, ethacrynic acid, bumetanide, furosemide, and torsemide. In some embodiments, the diuretic is selected from the group consisting of furosemide or torsemide.

In some embodiments the subject is also administered tafamidis.

### Pharmaceutical Compositions

Compound 1 can be prepared in various compositions suitable for delivery to a subject. A composition suitable for administration to a subject typically comprises Compound 1, or a pharmaceutically acceptable salt thereof and a pharmaceutically acceptable excipient.

The pharmaceutical compositions for the administration of Compound 1, or a pharmaceutically acceptable salt thereof can conveniently be presented in unit dosage form and can be prepared by any of the methods known in the art of pharmacy and drug delivery. All methods include the step of bringing the active ingredient into association with a carrier containing one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation.

Suitable formulations for use in the present invention are found in Remington: THE SCIENCE AND PRACTICE OF PHARMACY, 21st Ed., Gennaro, Ed., Lippincott Williams & Wilkins (2003), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

Compound 1 can be incorporated into a variety of formulations for therapeutic administration. More particularly, Compound 1 can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a compound of the present invention can be achieved in various ways, including oral, buccal, parenteral, intravenous, intradermal (e.g., subcutaneous, intramuscular), transdermal, etc., administration. Moreover, Compound 1 can be administered in a local rather than systemic manner, for example, in a depot or sustained release formulation.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

### Pharmaceutical Dosage Forms

The present disclosure includes pharmaceutical dosage forms of Compound 1, or a pharmaceutically acceptable form thereof. The dosage forms described herein are suitable for oral administration to a subject. The dosage form may be in any form suitable for oral administration, including, but not limited to, a capsule or a tablet.

In some embodiments, the present disclosure provides a single unit dosage capsule or tablet form containing 10-1,000 mg of Compound 1, having the formula:

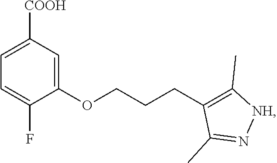 [SMILES: Cc1n[nH]c(C)c1CCCOc1cc(C(=O)O)ccc1F]

or a pharmaceutically acceptable salt thereof.

In some embodiments, the amount of Compound 1 is from about 100 to 800 mg. In some embodiments, the amount of Compound 1 is from about 150 to 600 mg. In some embodiments, the amount of Compound 1 is from about 200 to 400 mg. In some embodiments, the amount of Compound 1 is about 200 mg. In some embodiments, the amount of Compound 1 is about 400 mg. In some embodiments, the singe dosage capsule or tablet comprises the HCl salt of Compound 1, or a pharmaceutically acceptable salt thereof.

In some embodiments, the single unit dosage form of Compound 1 is a tablet.

In some embodiments, the single unit dosage form of Compound 1 is a capsule.

In some embodiments, the single unit dosage form is in a capsule of size #0, #1, #2, #3, #4, or #5. In some embodiments, the single unit dosage form is in a capsule of size #0. In some embodiments, the single unit dosage form is in a capsule of size #1. In some embodiments, the single unit dosage form is in a capsule of size #2. In some embodiments, the single unit dosage form is in a capsule of size #3. In some embodiments, the single unit dosage form is in a capsule of size #4. In some embodiments, the single unit dosage form is in a capsule of size #5.

### Kits

The disclosure also encompasses kits comprising pharmaceutical compositions and dosage forms of the invention.

In some aspects, the present invention provides a kit that includes Compound 1, or a phamectucially acceptable salt thereof. Some of the kits described herein include a label describing a method of administering Compound 1, or a pharmaceutically acceptable salt thereof. Some of the kits described herein include a label describing a method of treating transthyretin (TTR) amyloidosis. In some embodiments, the kits described herein include a label describing a method of treating Wild-type transthyretin amyloid cardiomyopathy (ATTR-CM, also called senile systemic amyloidosis). In some embodiments, the kits described herein include a label describing a method of treating familial amyloid cardiomyopathy (ATTR-mCM). In some embodiments, the kits described herein include a label describing a method of treating familial amyloid polyneuropathy (ATTR-PN, also called FAP).

The compositions of the present invention, including but not limited to, compositions comprising Compound 1 in a bottle, jar, vial, ampoule, tube, blister pack, or other container-closure system approved by the Food and Drug Administration (FDA) or other regulatory body, which may provide one or more unit dosages containing Compound 1, or a phamectucially acceptable salt thereof. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or one or more dosage units form including the formulation, and a notice or instructions describing a method of use as described herein.

Packaging systems such as blister packs include a thermoformable rigid film or PVC suitable for pharmaceutical packaging and a push through type lid. The lid can include a foil, made up of a primer/aluminum/heat-seal-coating, or may be paper based. A person of skill in the art will readily prepare blister packs comprising Compound 1, or a pharmaceutically acceptable salt thereof. Bottle systems described herein can be made in various sizes (e.g., 75 cc, 100 cc, 200 cc, etc), and generally include child resistant closures that can be made from polypropylene. In some embodiments, a pharmaceutical dosage form of Compound 1 is packaged in 75 cc bottles, with a child resistant closure. A person of skill in the art can readily prepare bottle systems described herein.

In some embodiments, the present disclosure provides kits for twice daily dosing. These kits provide one or more unit doses comprising Compound 1 for each administration.

In some embodiments, the total daily dose of Compound 1 is 800 mg, meaning 400 mg are administered at a first dosing, and 400 mg are administered at a second dosing. In some embodiments, two unit doses containing 200 mg of Compound 1 are administered at the first dosing and two unit doses containing 200 mg of Compound 1 are administered at the second dosing. In some embodiments, one unit dose containing 400 mg of Compound 1 is administered at the first dosing and one unit dose containing 400 mg of Compound 1 is administered the second dosing. In some embodiments, the HCl salt form of Compound 1 is administered.

In some embodiments, the total daily dose of Compound 1 is 1,600 mg, meaning 800 mg are administered at a first dosing, and 800 mg are administered at a second dosing. In some embodiments, four unit doses containing 200 mg of Compound 1 are administered at the first dosing and four unit doses containing 200 mg of Compound 1 are administered at the second dosing. In some embodiments, two unit doses containing 400 mg of Compound 1 is administered at the first dosing and two unit doses containing 400 mg of Compound 1 is administered the second dosing. In some embodiments, the HCl salt form of Compound 1 is administered.

### IV. Examples

The following examples are offered to illustrate, but not to limit, the claimed invention.

### Materials and Methods

### Fluorescent Probe Exclusion Assay (FPE)

Occupancy of AG10 in the thyroxine binding pocket of tetrameric TTR is determined by the ability of a fluorescent probe (Probe) to covalently bind to free tetrameric TTR binding sites in serum over a 6 hr reaction time. The fluorescent probe only fluoresces upon covalently binding to the ligand binding site of TTR. The presence of Acoramidis or Tafamidis in the binding site suppresses the development of fluorescent signal.

Aliquots of each serum sample are plated in 96 well plates. The fluorescence changes (λₑₓ=328 nm and λ_{cm}=384 nm) after addition of probe are monitored every 15 min using a fluorescent capable microplate for 6 hr at RT.

$$FPE \% {Stabilization}_{X} = 100 \times \frac{( {FPE}_{DMSO} @ 60 min ) - ( {FPE}_{X} @ 60 min )}{( {FPE}_{DMSO} @ 60 min )}$$

### Western Blot for Evaluating Stabilization of Tetrameric TTR

Stabilization of the tetramer of TTR by AG10 is determined by comparing the amount of tetrameric TTR protein remaining after acid denaturation for 72 hr to the initial amount of tetrameric TTR protein as determined by densitometric measurement of western blot gels.

Blood plasma samples from the subject are diluted with acidification buffer (sodium acetate, KCl, EDTA, DTT, pH about 4.0) for both the 0 and 72 hour time point. Time 0 hr samples are directly cross-linked with glutaraldehyde, and then quenched. 72 hr samples are incubated at room temperature for 72 hr and then cross-linked and quenched by the same protocol. All samples are then denatured by adding SDS gel loading buffer and boiled prior to gel loading. Each sample is separated in SDS-PAGE gels and analyzed by immunoblotting using anti-TTR antiserum (Polyclonal Rabbit Anti-Human Prealbumin. DAKO Cat #A0002). The density of all TTR bands are quantified using infrared LICOR imaging system or Fluorescence Imaging system and reported.

$$WB \% Stabilization = 100 \times \frac{72 hr TTR tetramer}{0 hr TTR tetramer}$$

### Example 1: Compound 1 (Acoramidis) Produces Near-Complete TTR Stabilization in Blood Samples from Patients with Certain Transthyretin Amyloidosis Mutations

Introduction: Transthyretin (TTR) amyloidosis (ATTR) is a progressive, fatal disease caused by destabilizing TTR variants (TTRv) and age-related factors. Dissociation of tetrameric TTR initiates protein misfolding, aggregation, and tissue deposition which constitutes the mechanism of disease. More destabilizing variants drive more severe clinical phenotypes. TTR stabilizers have demonstrated clinical benefits for neuropathic and cardiovascular outcomes correlated with the extent of TTR stabilization. Acoramidis (AG10) is a novel TTR stabilizer in development for the treatment of TTR amyloid cardiomyopathy.

Hypothesis: Acoramidis achieves near-complete in vitro TTR stabilization, exceeding levels achieved with clinically relevant concentrations of Tafamidis (a TTR stabilizer in clinical use), when added to blood samples from ATTRv patients across a spectrum of destabilizing TTR variants.

Methods: Two established assays assessed TTR stabilization: fluorescent probe exclusion (FPE; measures binding site occupancy), and Western blot (WB; quantifies tetrameric TTR persistence under conditions of accelerated dissociation). Examples of each assay are provided above. Over 60 individual patient samples representing 18 unique TTRv across a spectrum of intrinsic instability and clinical phenotypes were assayed. Acoramidis was added at μM, the target clinical steady-sate trough concentration is 8-10 μM, and compared to Tafamidis added at its clinical peak (26 μM) and trough (16 μM) concentrations reported for its maximal approved dose.

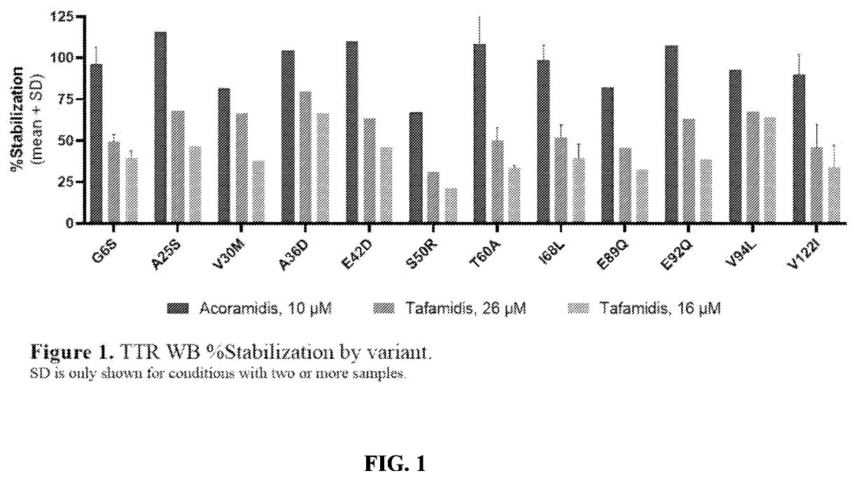

Results: Acoramidis bound serum TTR to a greater extent (103±13%) than either peak (87±14%) or trough (71±14%) concentrations of Tafamidis. WB assays showed that addition of Acoramidis resulted in significantly greater and more durable TTR stabilization (93±14%) than adding Tafamidis (peak: 49±14%, trough: 36±13%) in all paired individual patient plasma samples tested (FIG. 1, p<0.0001). Rare variants (A97S, D38A, F64L, L58H, P24S, Y114C) also demonstrated near complete stabilization upon in vitro addition of Acoramidis.

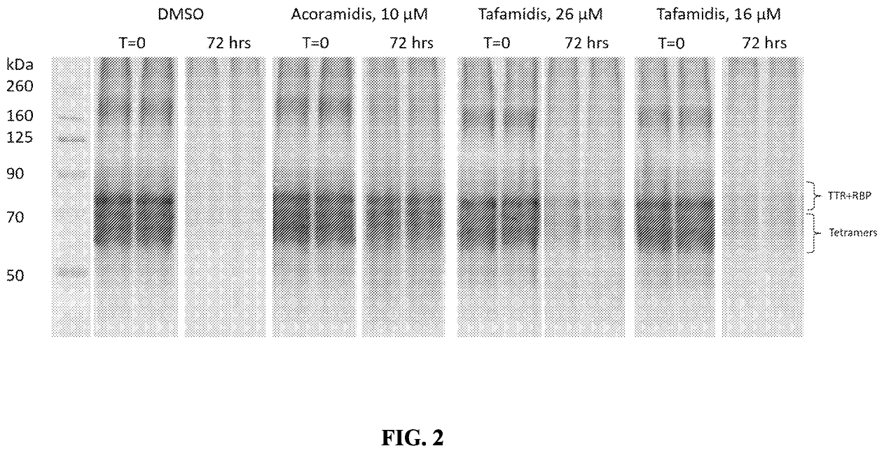

FIG. 2 shows a gel from the western blot assay of an individual V112I patient sample. All conditions were run in duplicate lanes. Brackets denoting the bands corresponding to tetrameric TTR with or without Retinol Binding Protein (RBP) are indicated.

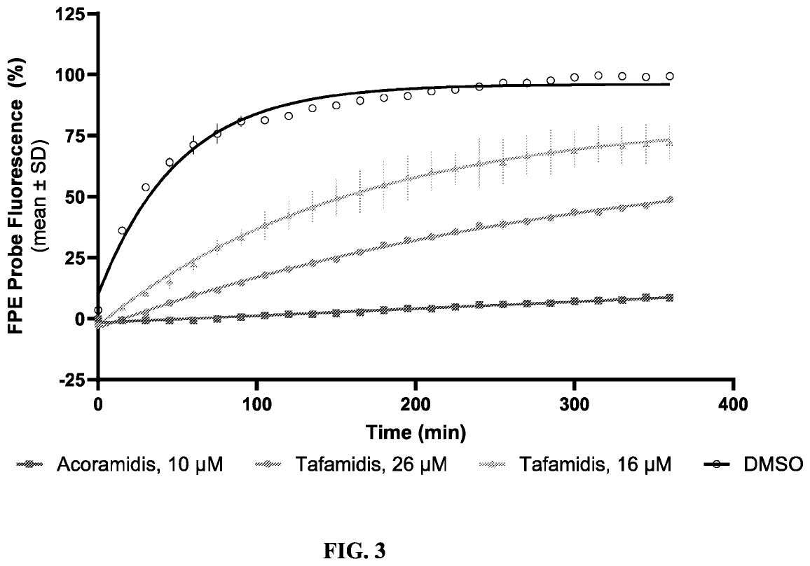

FIG. 3 shows an FPE time course of an indivudal with a V122I TTR mutation. The doses tested were 10 μM Acoramidis (filled squares); 26 μM Tafamidis (filled circles); 16 μM Tafamidis (filled triangles); and DMSO (open circles).

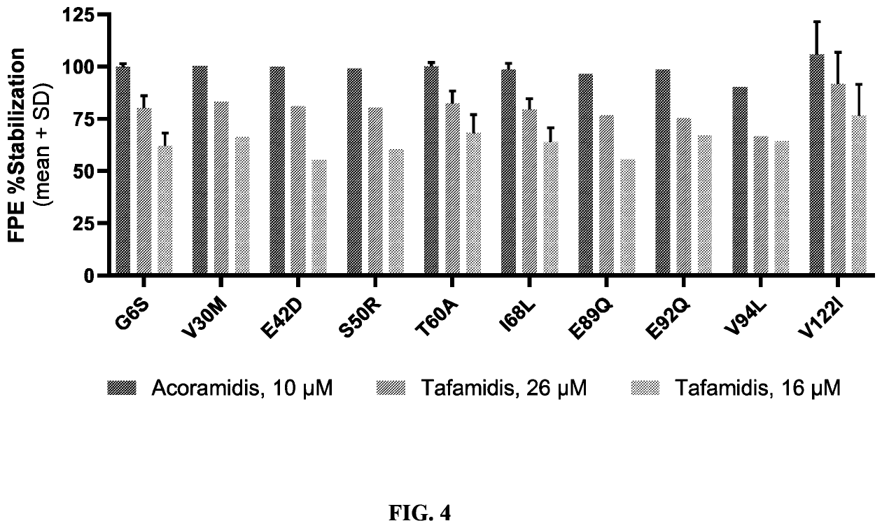

FIG. 4 shows the TTR FPE percent stabilization by variant. For each variant, the column on the left is 10 μM Acoramidis; the colum in the middle is 26 μM Tafamidis; and the column on the right is 16 μM Tafamidis.

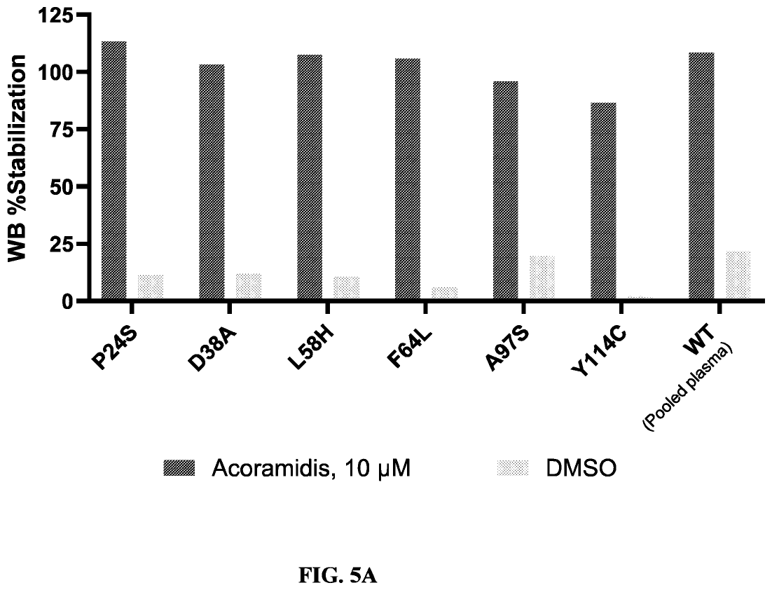

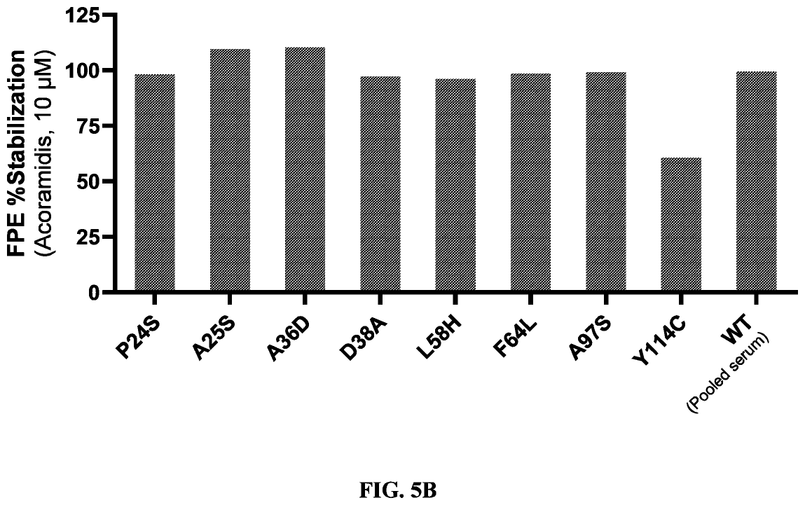

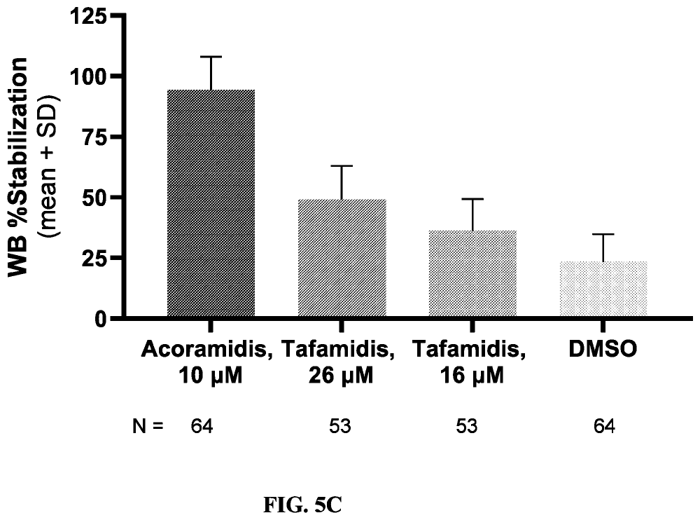

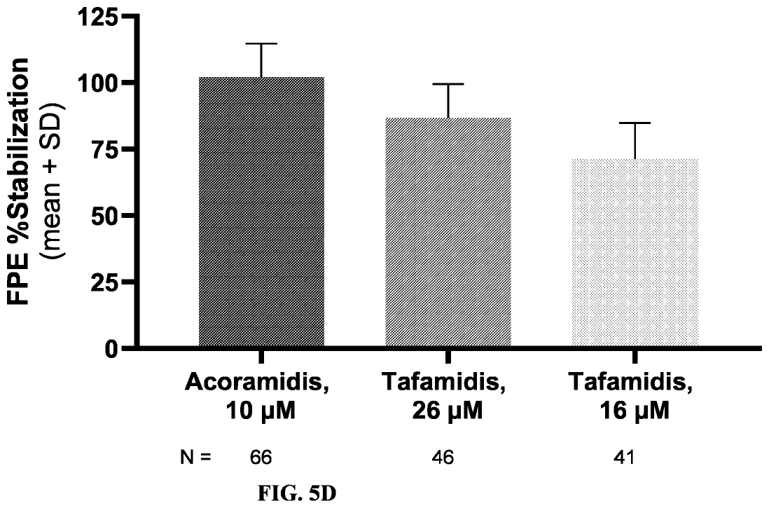

FIG. 5A-D shows stabilization of unique variant patient samples measured by (A) western blot (WB) and (B) fluorescent probe exclusion (FPE), with wildtype (WT) results as reference. Overall stabilization across all tested variant samples measured by (C) WB and (D) FPE.

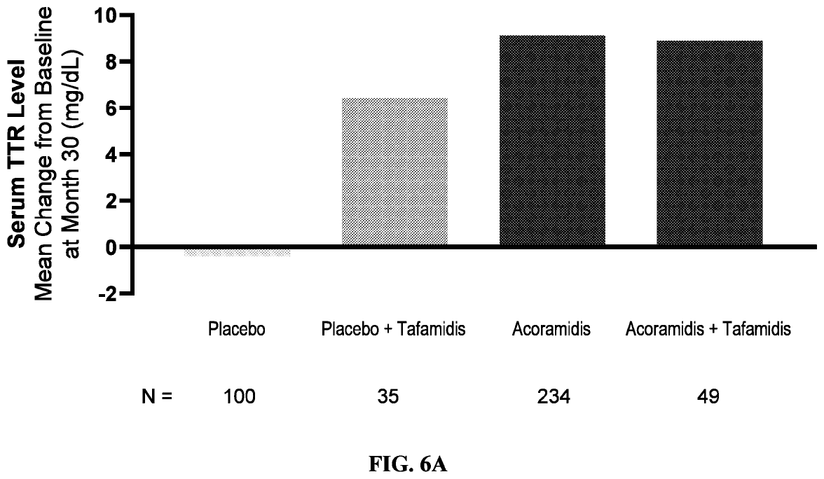

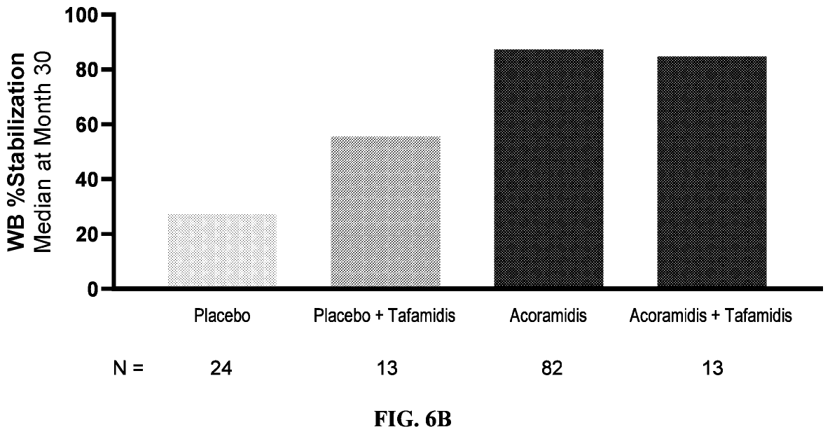

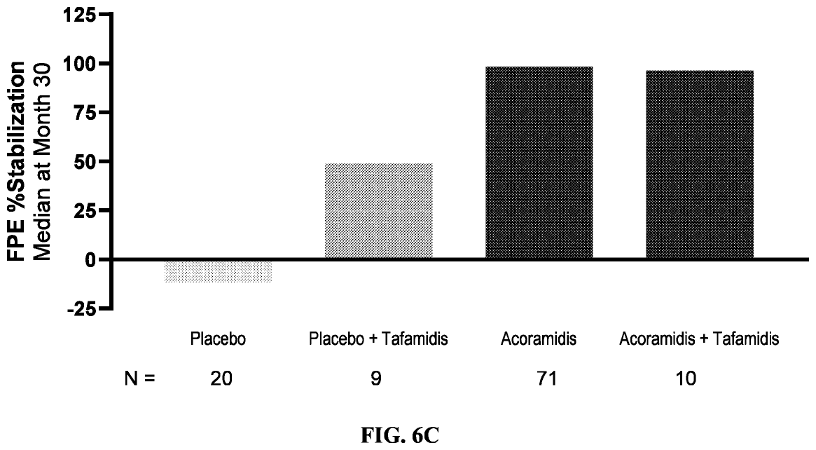

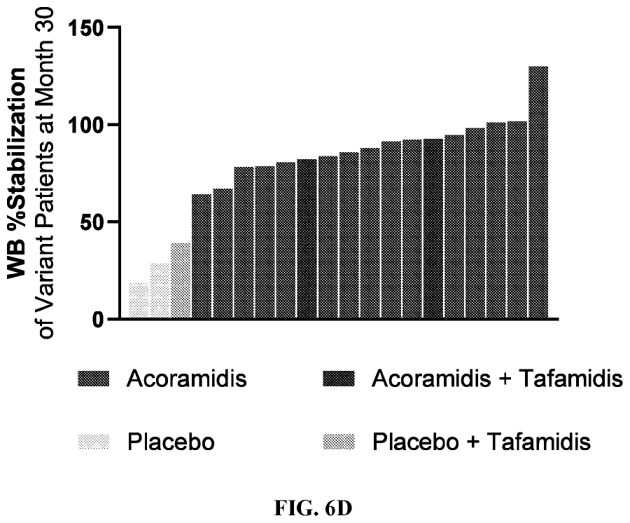

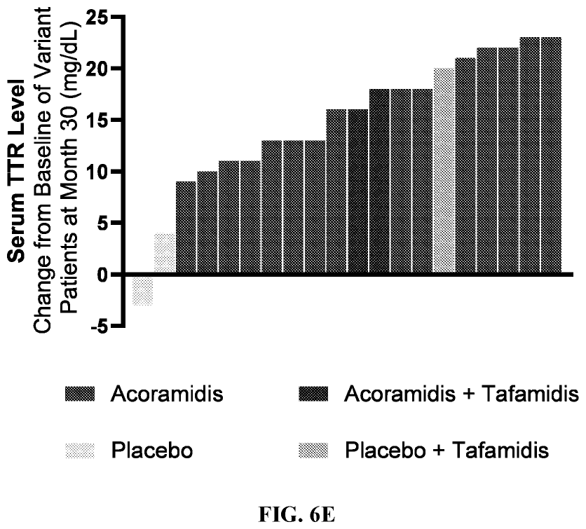

FIG. 6A-E shows Phase 3 ATTRibute-CM results in patients receiving Compound 1 in a total daily dose of 1,600 mg (800 mg b.i.d.) or placebo. The reults demonstrate that ex vivo TTR stabilization correlates with in vivo measurement of serum TTR. (A) shows mean change from baseline in serum TTR at month 30; (B) shows median of WB % stabilization at month 30; (C) shows median of FPE % stabilization at month 30; (D) is a waterfall plot of variant patients WB % stabilization month 30; and (E) is a waterfall plot of variant patients change from baseline 5 in serum TTR at month 30. Note: variant=mutant TTR genotype. The Acoramidis+Tafamidis columns show data from patients receiving both compounds. This shows that addition of Tafamidis does not increase TTR stabilization in patients receiving Acoramidis, while also showing that addition of acoradmidis increases TTR stabilization in patients receiving Tafamidis.

Table 1 and Table 2, below summarize the WB and FPE results.

TABLE 1
Summary of head-to-head WB % Stabilization Results

| | WB Mean % Stabilization (SD) | | | | | |
|---|---|---|---|---|---|---|
| | | Acoramidis, | | Tafamidis, | Tafamidis, | |
| Variant | N | 10 μM | | 26 μM | 16 μM | DMSO |
| G6S | 2 | 95.93 | (10.67) | 49.35 (4.37) | 39.16 (4.59) | 28.27 (0.13) |
| A25S | 1 | 115.43 | | 68.06 | 46.47 | 20.88 |
| V30M | 1 | 81.73 | | 66.51 | 37.47 | 31.95 |
| A36D | 1 | 104.20 | | 79.70 | 66.29 | 50.54 |
| E42D | 1 | 109.63 | | 63.57 | 46.08 | 24.14 |
| S50R | 1 | 67.00 | | 31.00 | 21.00 | 12.00 |
| T60A | 3 | 108.51 | (25.51) | 49.64 (8.33) | 33.44 (1.60) | 23.41 (6.58) |
| I68L | 7 | 98.53 | (9.39) | 51.6 (8.00) | 39.34 (8.59) | 21.38 (7.61) |
| E89Q | 1 | 82.23 | | 45.26 | 32.16 | 26.96 |
| E92Q | 1 | 107.21 | | 62.76 | 38.49 | 25.89 |
| V94L | 1 | 92.79 | | 67.37 | 64.19 | 25.96 |
| V122I | 33 | 89.70 | (12.33) | 45.73 (14.13) | 33.93 (13.39) | 25.50 (12.97) |
| Variant | 53 | 92.97 | (13.97) | 49.10 (13.89) | 36.34 (12.88) | 25.21 (11.46) |
| mean | | | | | | |

Note:
SD is only shown for conditions with 2 or more samples. Overall mean WB % Stabilization by 10 μM Acoramidis is significantly higher than 26 μM Tafamidis (p < 0.0001).

TABLE 2
Summary of head-to-head FPE % Stabilization Results

| | FPE Mean % Stabilization (SD) | | | |
|---|---|---|---|---|
| | | Acoramidis, | Tafamidis, | Tafamidis, |
| Variant | N | 10 μM | 26 μM | 16 μM |
| G6S | 2 | 99.81 (1.55) | 80.02 (5.99) | 61.96 (6.12) |
| V30M | 1 | 100.34 | 83.15 | 66.32 |
| E42D | 1 | 100.05 | 80.99 | 55.28 |
| S50R | 1 | 99.11 | 80.27 | 60.34 |
| T60A | 4 | 100.12 (1.77) | 82.32 (6.05) | 68.10 (8.92) |
| I68L | 5 | 98.55 (2.98) | 79.4 (5.28) | 63.95 (6.73) |
| E89Q | 1 | 96.44 | 76.66 | 55.48 |
| E92Q | 1 | 98.46 | 75.41 | 66.97 |
| V94L | 1 | 90.13 | 66.46 | 64.22 |
| V122I | 24 | 105.76 (15.65) | 91.62 (15.18) | 76.54 (14.83) |
| Variant | 41 | 102.82 (12.52) | 86.54 (13.51) | 71.26 (13.58) |
| mean | | | | |
| Pooled | 6* | 99.37 (1.77) | 64.69 (5.52) | 48.61 (6.43) |
| serum | | | | |
| (WT) | | | | |

Note:
SD is only shown for conditions with 2 or more samples. Overall mean FPE % Stabilization by 10 μM Acoramidis is significantly higher than 26 μM Tafamidis (p < 0.0001).
*Healthy pooled serum was tested using 6 replicates per assay condition.

Conclusions: At its target therapeutic trough concentration, Acoramidis achieved near-complete TTR stabilization across 18 unique genotypes; in the subset of paired samples, the stabilization was significantly greater for Acoramidis than for Tafamidis even at its peak clinical concentration. This observation held across a range of destabilizing mutations, including a~2-fold greater stabilization than Tafamidis for the prevalent cardiomyopathie V122I variant. Based on the mechanism of disease and the association between TTR destabilization and severity of clinical outcomes, these data suggest that Acoramidis has the potential to be a clinically differentiated and efficacious treatment option for patients with ATTRv, independent of variant genotype.

Results from the positive randomized, controlled Phase 3 ATTRibute-CM study are consistent with in vitro findings. Acoramidis achieved near-complete stabilization in WT and ATTRv patients at month 30 as measured by ex vivo WB and FPE assays. Acoramidis achieved a greater degree of TTR stabilization as compared to clinically relevant concentrations of Tafamidis, independent of TTR genotype. In vitro and in vivo assessments of TTR stabilization demonstrated Acoramidis activity across 18 unique TTR variants encountered.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating transthyretin (TTR) amyloidosis in a subject in need thereof, the method comprising administering to a subject in need thereof a therapeutically effective amount of Compound 1, having the formula: 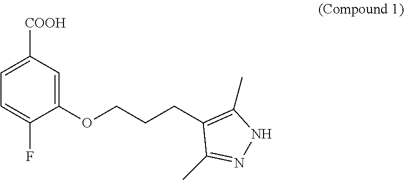 [SMILES: Cc1n[nH]c(C)c1CCCOc1cc(C(=O)O)ccc1F]
or a pharmaceutically acceptable salt thereof, wherein said therapeutically effective amount is a total daily dosage of about 10 milligrams (mg) to about 2,000 mg of Compound 1 in HCl salt form or an equivalent amount of Compound 1 in free base form or in a different salt form, and
wherein the TTR amyloidosis in said subject is characterized by a TTR protein comprising a mutation selected from the group consisting of:
a glycine to serine mutation at position 6 (G6S),
an alanine to serine mutation at position 25 (A25S),
an alanine to aspartic acid mutation at position 36 (A36D),
a glutamic acid to aspartic acid mutation at position 42 (E42D),
a serine to arginine mutation at position 50 (S50R),
an isoleucine to leucine mutation at position 68 (168L),
a glutamic acid to glutamine mutation at position 89 (E89Q),
a glutamic acid to glutamine mutation at position 92 (E92Q),
a valine to leucine mutation at position 94 (V94L), and
an alanine to serine mutation at position 97 (A97S).

2. The method of claim 1, wherein said therapeutically effective amount is a total daily dosage of about 10 mg to about 50 mg of Compound 1 in HCl salt form or an equivalent amount of Compound 1 in free base form or in a different salt form.

3. The method of claim 1, wherein said therapeutically effective amount is a total daily dosage of about 50 mg to about 300 mg of Compound 1 in HCl salt form or an equivalent amount of Compound 1 in free base form or in a different salt form.

4. The method of claim 1, wherein said therapeutically effective amount is a total daily dosage of about 50 mg to about 150 mg of Compound 1 in HCl salt form or an equivalent amount of Compound 1 in free base form or in a different salt form.

5. The method of claim 1, wherein said therapeutically effective amount is a total daily dosage of about 150 mg to about 800 mg of Compound 1 in HCl salt form or an equivalent amount of Compound 1 in free base form or in a different salt form.

6. The method of claim 1, wherein said therapeutically effective amount is a total daily dosage of about 800 mg to about 1,600 mg of Compound 1 in HCl salt form or an equivalent amount of Compound 1 in free base form or in a different salt form.

7. The method of claim 1, wherein said therapeutically effective amount is a total daily dosage of about 800 mg of Compound 1 in HCl salt form or an equivalent amount of Compound 1 in free base form or in a different salt form.

8. The method of claim 1, wherein said therapeutically effective amount is a total daily dosage of about 1,600 mg of Compound 1 in HCl salt form or an equivalent amount of Compound 1 in free base form or in a different salt form.

9. The method of claim 1, wherein said therapeutically effective amount is a total daily dosage of about 1,100 mg to about 1,300 mg of Compound 1 in HCl salt form or an equivalent amount of Compound 1 in free base form or in a different salt form.

10. The method of claim 1, wherein said therapeutically effective amount is a total daily dosage of about 1,236 mg of Compound 1 in HCl salt form or an equivalent amount of Compound 1 in free base form or in a different salt form.

11. The method of any one of claims 1 to 10, wherein Compound 1 is in HCl salt form.

12. The method of any one of claims 1 to 11, wherein Compound 1 is administered orally.

13. The method of any one of claims 1 to 12, wherein Compound 1 is administered once daily.

14. The method of any one of claims 1 to 12, wherein Compound 1 is administered twice daily.

15. The method of any one of claims 1 to 12, wherein Compound 1 is administered three times daily.

16. The method of any one of claims 1 to 12, wherein Compound 1 is administered four times daily.

17. The method of any one of claims 1 to 16, wherein the TTR amyloidosis in said subject is characterized by a TTR protein comprising a glycine to serine mutation at position 6 (G6S).

18. The method of any one of claims 1 to 16, wherein the TTR amyloidosis in said subject is characterized by a TTR protein comprising an alanine to serine mutation at position 25 (A25S).

19. The method of any one of claims 1 to 16, wherein the TTR amyloidosis in said subject is characterized by a TTR protein comprising an alanine to aspartic acid mutation at position 36 (A36D).

20. The method of any one of claims 1 to 16, wherein the TTR amyloidosis in said subject is characterized by a TTR protein comprising a glutamic acid to aspartic acid mutation at position 42 (E42D).

21. The method of any one of claims 1 to 16, wherein the TTR amyloidosis in said subject is characterized by a TTR protein comprising a serine to arginine mutation at position 50 (S50R).

22. The method of any one of claims 1 to 16, wherein the TTR amyloidosis in said subject is characterized by a TTR protein comprising an isoleucine to leucine mutation at position 68 (168L).

23. The method of any one of claims 1 to 16, wherein the TTR amyloidosis in said subject is characterized by a TTR protein comprising a glutamic acid to glutamine mutation at position 89 (E89Q).

24. The method of any one of claims 1 to 16, wherein the TTR amyloidosis in said subject is characterized by a TTR protein comprising a glutamic acid to glutamine mutation at position 92 (E92Q).

25. The method of any one of claims 1 to 16, wherein the TTR amyloidosis in said subject is characterized by a TTR protein comprising a valine to leucine mutation at position 94 (V94L).

26. The method of any one of claims 1 to 16, wherein the TTR amyloidosis in said subject is characterized by a TTR protein comprising an alanine to serine mutation at position 97 (A97S).

27. The method of any one of claims 1 to 26, wherein the TTR amyloidosis is a disease or condition selected from the group consisting of familial amyloid polyneuropathy, familial amyloid cardiomyopathy, senile systemic amyloidosis, central amyloidosis, ocular amyloidosis, Leptomeningeal amyloidosis, oculoleptomeningeal amyloidosis, vitreous amyloidosis, gastrointestinal amyloidosis, neuropathic amyloidosis, non-neuropathic amyloidosis, non-hereditary amyloidosis, reactive/secondary amyloidosis, cerebral amyloidosis.

28. The method of any one of claims 1 to 26, wherein the TTR amyloidosis is transthyretin amyloidosis (ATTR) cardiomyopathy or transthyretin amyloidosis (ATTR) polyneuropathy.

29. The method of any one of claims 1 to 26, wherein the TTR amyloidosis is transthyretin amyloidosis (ATTR) cardiomyopathy.

30. The method of claim 29, wherein said ATTR cardiomyopathy is familial ATTR cardiomyopathy (ATTRm-CM).

31. The method of claim 29 or 30, wherein administration of a therapeutically effective amount of Compound 1, or a pharmaceutically acceptable salt thereof, in a subject with ATTR cardiomyopathy improves the subject's risk of mortality, morbidity, and quality of life.

32. The method of any one of claims 29 to 31, wherein administration of a therapeutically effective amount of Compound 1, or a pharmaceutically acceptable salt thereof, in a subject with ATTR cardiomyopathy improves the subject's performance in a six minute walk test (6MWT).

33. The method of claim 32, wherein the subject walks at least 25 meters (m) further than a baseline distance measured prior to treatment with Compound 1, or a pharmaceutically acceptable salt thereof.

34. The method of any one of claims 29 to 33, wherein administration of a therapeutically effective amount of Compound 1, or a pharmaceutically acceptable salt thereof, in a subject with ATTR cardiomyopathy decreases mortality as compared to subjects not receiving treatment.

35. The method of claim 34, wherein administration of a therapeutically effective amount of Compound 1, or a pharmaceutically acceptable salt thereof, in a subject with ATTR cardiomyopathy decreases all cause mortality as compared to subjects not receiving treatment.

36. The method of any one of claims 29 to 35, wherein administration of a therapeutically effective amount of Compound 1, or a pharmaceutically acceptable salt thereof, in a subject with ATTR cardiomyopathy improves, stabilizes or delays worsening in New York Heart Association (NYHA) functional classification of subjects.

37. The method of claim 36, wherein administration of a therapeutically effective amount of Compound 1, or a pharmaceutically acceptable salt thereof, reduces the NYHA functional classification of the subject.

38. The method of claim 37, wherein the NYHA functional classification is reduced from class IV to class III, from class IV to class II, or from class IV to class I.

39. The method of claim 37, wherein the NYHA functional classification is reduced from class III to class II.

40. The method of claim 37, wherein the NYHA functional classification is reduced from class III to class I.

41. The method of claim 37, wherein the NYHA functional classification is reduced from class II to class I.

42. The method of any one of claims 29 to 41, wherein administration of a therapeutically effective amount of Compound 1, or a pharmaceutically acceptable salt thereof, in a subject with ATTR cardiomyopathy improves, stabilizes or delays worsening in the Kansas City Cardiomyopathy Questionnaire (KCCQ) classification of subjects.

43. The method of claim 42, wherein subjects have an average improvement of at least one level in the KCCQ classification of subjects.

44. The method of any one of claims 29 to 43, wherein administration of a therapeutically effective amount of Compound 1, or a pharmaceutically acceptable salt thereof, in a subject with ATTR cardiomyopathy improves, stabilizes or delays worsening in the EuroQoL-5 Dimensions (EQ-5D-5L) classification of subjects.

45. The method of claim 44, wherein subjects have an average improvement of at least five points in the EQ-5D-5L utility score.

46. The method of any one of claims 29 to 45, wherein administration of a therapeutically effective amount of Compound 1, or a pharmaceutically acceptable salt thereof, in a subject with ATTR cardiomyopathy decreases serum blood levels of brain natriuretic peptide (BNP).

47. The method of claim 46, wherein serum blood levels of BNP of said subject decreases at least 10% as compared to a baseline level of BNP in said subject prior to treatment with Compound 1, or a pharmaceutically acceptable salt thereof.

48. The method of any one of claims 29 to 47, wherein administration of a therapeutically effective amount of Compound 1, or a pharmaceutically acceptable salt thereof, in a subject with ATTR cardiomyopathy decreases serum blood levels of N-terminal pro-brain natriuretic peptide (N-terminal pro-BNP).

49. The method of claim 48, wherein serum blood levels of N-terminal pro-BNP of said subject decreases at least 10% as compared to a baseline level of N-terminal pro-BNP in said subject prior to treatment with Compound 1, or a pharmaceutically acceptable salt thereof.

50. The method of any one of claims 29 to 49, wherein administration of a therapeutically effective amount of Compound 1 in a subject with ATTR cardiomyopathy decreases the frequency of cardiovascular-related hospitalizations as compared to subjects not receiving treatment.

51. The method of any one of claims 1 to 26, wherein said TTR amyloidosis is transthyretin amyloidosis (ATTR) polyneuropathy.

52. The method of claim 51, wherein said ATTR polyneuropathy is familial ATTR polyneuropathy (ATTRm-PN).

53. The method of claim 51 or 52, wherein administration of a therapeutically effective amount of Compound 1, or a pharmaceutically acceptable salt thereof, in a subject with ATTR polyneuropathy improves the Neuropathy Impairment Score (NIS) in said subject.

54. The method of claim 53, wherein the NIS score is reduced by at least 10% as compared to a baseline level measured prior to treatment with Compound 1, or a pharmaceutically acceptable salt thereof.

55. The method of any one of claims 51 to 54, wherein administration of a therapeutically effective amount of Compound 1, or a pharmaceutically acceptable salt thereof, in a subject with ATTR polyneuropathy improves the modified Neuropathy Impairment Score+7 (mNIS+7) in said subject.

56. The method of claim 55, wherein the mNIS+7 score is reduced by at least 10% as compared to a baseline level measured prior to treatment with Compound 1, or a pharmaceutically acceptable salt thereof.

57. The method of any one of claims 51 to 56, wherein administration of a therapeutically effective amount of Compound 1, or a pharmaceutically acceptable salt thereof, in a subject with ATTR polyneuropathy improves the subject's Norfolk Quality of Life Diabetic Neuropathy (QOL-DN) questionnaire score.

58. The method of claim 57, wherein the Norfolk QOL-DN score is improved by at least 10% as compared to a baseline level measured prior to treatment with Compound 1, or a pharmaceutically acceptable salt thereof.

59. The method of any one of claims 51 to 58, wherein administration of a therapeutically effective amount of Compound 1, or a pharmaceutically acceptable salt thereof, in a subject with ATTR polyneuropathy improves the subject's composite autonomic symptom score (COMPASS-31) score.

60. The method of claim 59, wherein the COMPASS-31 score is improved by at least 0.5 points as compared to a baseline level measured prior to treatment with Compound 1, or a pharmaceutically acceptable salt thereof.

61. The method of any one of claims 51 to 60, wherein administration of a therapeutically effective amount of Compound 1 in a subject with ATTR polyneuropathy improves the modified BMI (mBMI) of the subject.

62. The method of any one of claims 51 to 61, wherein administration of a therapeutically effective amount of Compound 1, or a pharmaceutically acceptable salt thereof, in a subject with ATTR polyneuropathy improves the ten-meter walk test (10MWT) speed of the subject.

63. The method of any one of claims 1 to 62, wherein Compound 1, or a pharmaceutically acceptable salt thereof, is administered chronically.

64. The method of any one of claims 1 to 63, wherein Compound 1, or a pharmaceutically acceptable salt thereof, is administered for at least 28 days.

65. The method of any one of claims 1 to 64, wherein Compound 1, or a pharmaceutically acceptable salt thereof, is administered for at least 56 days.

66. The method of any one of claims 1 to 65, wherein Compound 1, or a pharmaceutically acceptable salt thereof, is administered for at least 84 days.

67. The method of any one of claims 1 to 66, wherein Compound 1, or a pharmaceutically acceptable salt thereof, is administered for at least 18 months.

68. The method of any one of claims 1 to 67, wherein Compound 1, or a pharmaceutically acceptable salt thereof, is administered for at least 30 months.

69. The method of any one of claims 1 to 68, wherein the subject is receiveing a diuretic therapy agent.

70. The method of claim 69, wherein the diuretic therapy agent is furosemide or torsemide.

71. The method of any one of claims 1 to 70, wherein the subject is also administered tafamidis.
